(19) United States
(12) Patent Application Publication
(10) Pub. No.: US 2026/0345105 A1
(43) Pub. Date: Nov. 5, 2026

(54) METHODS, SYSTEMS, AND COMPUTER READABLE MEDIA FOR INTRA-OPERATIVE ASSESSMENT OF TISSUE SPECIMEN MAMMOGRAPHY IMAGES TO PREDICT TUMOR MARGIN STATUS

(71) Applicant: THE UNIVERSITY OF NORTH CAROLINA AT CHAPEL HILL, Chapel Hill, NC (US)

(72) Inventors: Kevin Andrew Chen, Durham, NC (US); Shawn Michael Gomez, Durham, NC (US); Kristalyn Kay Gallagher, Chapel Hill, NC (US)

(21) Appl. No.: 19/123,661

(22) PCT Filed: Oct. 23, 2023
(86) PCT No.: PCT/US2023/035719

§ 371 Date: Apr. 23, 2025

Related U.S. Application Data
(60) Provisional application No. 63/418,592, filed on Oct. 23, 2022.

(51) Int. Cl. A61B 6/00; A61B 6/02; A61B 6/12; A61B 6/50

(52) U.S. Cl. CPC A61B 6/5217; A61B 6/025; A61B 6/12; A61B 6/502

(57) ABSTRACT

A method for intra-operative assessment of a tissue specimen mammogram to predict tumor margin status includes receiving, during breast-conserving surgery, a mammogram of a resected breast tissue specimen. The method further includes providing the mammogram as input to a tumor margin classifier comprising a trained machine learning model. The method further includes GPU MEM producing, based on the input, intra-operatively, in real time, and using the trained machine learning model, a prediction of whether the specimen has a positive tumor margin status or a negative tumor margin status. The method further includes outputting the prediction intra-operatively.

### RELATED APPLICATIONS

This application claims the priority benefit of U.S. Provisional Patent Application No. 63/418,592, filed Oct. 23, 2022, the disclosure of which is incorporated herein by reference in its entirety.

### TECHNICAL FIELD

The subject matter described herein relates to intra-operative assessment of breast tissue specimens in human subjects. More particularly, the subject matter described herein relates to methods, systems, and computer readable media for intra-operative assessment of tissue specimen mammography images to predict tumor margin status.

### BACKGROUND

During breast-conserving surgery to remove cancerous lesions, the surgeon removes portions of breast tissue believed to contain cancerous lesions as well as a margin of healthy tissue around the cancerous lesion. After removal, the samples are evaluated by pathology for the microscopic presence or absence of cancer cells at the edges of each slice. In most cases, the samples are not evaluated intra-operatively and are instead evaluated in a pathology lab days or weeks later to determine margin status. A positive margin status means that the sample being evaluated has cancer cells at or near (the amount of allowed margin depends on the cancer type) the edge of the tissue sample. A negative margin status means there are no cancer cells present at or near the edge of the tissue sample.

One problem with evaluating samples after surgery is that if a positive margin is detected, additional surgery may be recommended as a treatment option. Requiring patients to undergo multiple surgeries is undesirable.

Margin status can be evaluated intra-operatively, but typically requires the presence of on-demand laboratory facilities that can evaluate tissue samples intra-operatively. While some larger treatment centers may have laboratory facilities and resources to evaluate tumor margin status intra-operatively, such facilities may not be present in all treatment centers. As a result, intra-operative assessment of margin status in some cases depends on intra-operative specimen mammography, which is an X-ray of the removed tissue. This is then interpreted by the surgeon, who may or may not specialize in breast-conserving surgery. Due to the lack of expertise at some treatment centers and the inability to definitively evaluate tumor margins intra-operatively, there exists a need for improved methods, systems, and computer readable media for intra-operative assessment of tissue specimen mammography images to predict tumor margin status.

### SUMMARY

A method for intra-operative assessment of a tissue specimen mammogram to predict tumor margin status includes receiving, during breast-conserving surgery, a mammogram of a resected breast tissue specimen. The method further includes providing the mammogram as input to a tumor margin classifier comprising a trained machine learning model. The method further includes producing, based on the input, intra-operatively, in real time, and using the trained machine learning model, a prediction of whether the specimen has a positive tumor margin status or a negative tumor margin status. The method further includes outputting the prediction intra-operatively.

According to another aspect of the subject matter described herein, the method includes using the prediction to determine whether to resect additional breast tissue.

According to another aspect of the subject matter described herein, the trained machine learning model comprises an image classifier model pre-trained based on non-mammography images.

According to another aspect of the subject matter described herein, the trained machine learning model comprises at least one dense layer connected to the image classifier model.

According to another aspect of the subject matter described herein, weights assigned to neurons in the at least one dense layer result from initial training of the at least one dense layer using specimen mammograms and expert margin status classifications based on pathology as inputs with the image classifier model frozen.

According to another aspect of the subject matter described herein, the weights assigned to neurons in the at least one dense layer additionally result from training of the at least one dense layer in combination with the image classifier model and with the image classifier model unfrozen.

According to another aspect of the subject matter described herein, the trained machine learning model comprises at least one dropout layer following the at least one dense layer.

According to another aspect of the subject matter described herein, the prediction of whether the specimen has a positive tumor margin status or a negative tumor margin status comprises a probability that the specimen has a positive tumor margin status.

According to another aspect of the subject matter described herein, the prediction of whether the specimen has a positive tumor margin status or a negative tumor margin status comprises a probability that the specimen has a negative tumor margin status.

According to another aspect of the subject matter described herein, the trained machine learning model is configured to generate the prediction of whether the specimen has a positive tumor margin status or a negative tumor margin status based on a least one input in addition to the mammogram.

According to another aspect of the subject matter described herein, a system for intra-operative assessment of a tissue specimen mammogram to predict tumor margin status is provided. The system includes a computing platform including at least one processor and a memory. The system further includes a tumor margin classifier comprising a trained machine learning model implemented by the at least one processor for receiving as input during breast-conserving surgery, a mammogram of a resected breast tissue specimen, producing, based on the input, intra-operatively, in real time, and using the trained machine learning model, a prediction of whether the specimen has a positive tumor margin status or a negative tumor margin status, and outputting the prediction intra-operatively.

According to another aspect of the subject matter described herein, a non-transitory computer readable medium having stored thereon executable instructions that when executed by a processor of a computer control the computer to perform steps is provided. The steps include receiving, during breast-conserving surgery, a mammogram of a resected breast tissue specimen. The steps further include providing the mammogram as input to a tumor margin classifier comprising a trained machine learning model. The steps further include producing, based on the input, intra-operatively, in real time, and using the trained machine learning model, a prediction of whether the specimen has a positive tumor margin status or a negative tumor margin status. The steps further include outputting the prediction intra-operatively.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Exemplary implementations of the subject matter described herein will now be explained with reference to the accompanying drawings, of which:

FIG. 1 illustrates example specimen mammograms classified by margin status;

FIG. 2A is a flow diagram illustrating an example of mammogram classification, training of a machine learning model using the classified mammograms, and evaluation of the trained machine learning model using a test set of classified images;

FIG. 2B is a block diagram of a machine learning model for intra-operative assessment of tumor margin status;

FIG. 3 is a confusion matrix illustrating predictive ability of the trained Xception model for positive and negative margin statuses;

FIG. 4 illustrates receiver operating characteristics curves for the trained VGG19, Xception, and Resnet150v2 models;

FIG. 5 is an image illustrating results of pixel importance analysis as evaluated by GradCam for the trained Xception model;

FIG. 6 is a block diagram illustrating an exemplary computing platform with a tumor margin classifier that implements as trained machine learning model;

FIG. 7 is a flow chart illustrating an exemplary process for intra-operative assessment of tumor margin status using a trained machine learning model; and

FIG. 8 is a diagram of a proposed model for predicting tumor margin status from 3D tomosynthesis data.

### DETAILED DESCRIPTION

### Introduction

In breast conserving surgery for breast cancer, obtaining negative margins is critical to reduce recurrence. Re-operation rates for positive margins after partial mastectomy remains high, at around 22%.¹ Multiple intra-operative interventions have been applied to reduce the rate of positive margins, including intra-operative specimen mammography. However, specimen mammography can be inaccurate, with sensitivity ranging from 20% to 58%.²⁻⁵ A tool to improve the accuracy and reliability of specimen mammography for partial mastectomy would be helpful to reduce the rate of re-operation.

Deep learning has been extensively applied to mammography, with a recent systematic review identifying 48 studies which attempted to diagnose breast cancer from mammography. Deep learning also shows promise for intra-operative use, with recent applications to the interpretation of laparoscopic video.⁶⁻⁸ However, deep learning has not yet been applied to specimen mammography.

The goal of this study is to develop a model which can accurately predict margin positivity for partial mastectomy based on specimen mammography alone. We hypothesized that a prototype created with a single-institution dataset could match previously published accuracy metrics.

### Methods

### Dataset

Prior to data collection, IRB approval was obtained. From July 2017 to June 2020, specimen mammograms were prospectively collected as part of a surgeon's quality assurance process. Using retrospective chart review, specimen mammograms were matched with pathology reports and categorized into “positive” and “negative” classes based on NCCN guidelines. ⁹ For specimens with invasive cancer, a positive margin was defined as “ink on tumor.” For specimens with ductal carcinoma in situ (DCIS), a positive margin was defined as DCIS within 2 mm of the margin. For specimens containing both DCIS and invasive cancer, but with DCIS within 2 mm of the margin, the classification was unclear. Because DCIS in this situation is unlikely to appear differently compared with DCIS alone, we elected to categorize this pathology as “positive,” while acknowledging that, clinically, this result is treated as “negative.” After data collection, the images were censored of all identifying information.

### Data Processing

The dataset was divided randomly into training and validation sets in a 80/20 ratio. Images were resized to 512×512 pixels. Pixel intensity was normalized by dividing by 255. The training set underwent standard data augmentation including random flipping (vertically and horizontally), zoom, shifts (horizontal and vertical), and rotation.¹⁰

Model architecture and training

We compared the performance of three widely used architectures for transfer learning including Xception, Resnet-152, and VGG19.¹¹ Four additional fully-connected layers were added to each of these models, with 3 dropout layers (0.3) in between. The fully-connected layers had 512, 256, 128, and 1 nodes each. Each dense layer used the “ReLu” activation except the output layer, which used a sigmoid activation. Model training was performed in two sequences. First, all layers of the original architecture were held frozen and the new dense layers were trained with a higher learning rate of 1*10^{∧}-4. Second, all layers were unfrozen, and the model was trained at a lower learning rate of 1*10^{∧}-6. Early stopping was used to avoid training set overfitting, with a minimum change of 1*10^{∧}-4 and a patience of 5 epochs.¹²

Model evaluation

The model's accuracy was assessed using balanced accuracy, sensitivity, specificity, positive predictive value, negative predictive value, and area under the receiver operating characteristic curve. In addition, GradCam was used to create saliency maps to assess pixel importance for the highest performing model.¹³

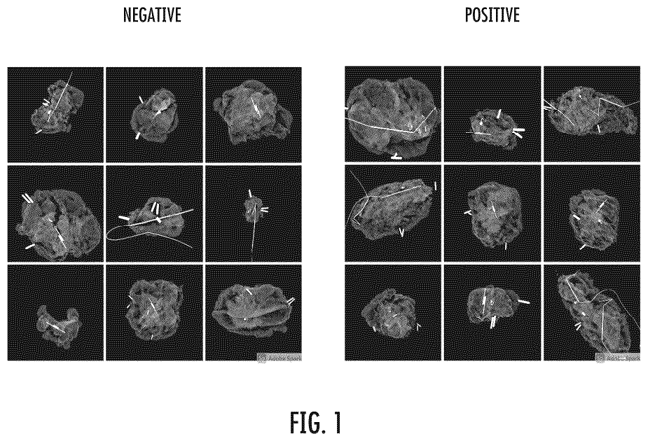

FIG. 1 illustrates example specimen mammograms classified by margin status class. In particular, the left hand mammograms in FIG. 1 illustrate specimen mammograms classified as having a negative margin status. The right hand mammograms in FIG. 1 illustrate specimen mammograms classified as having a positive margin status.

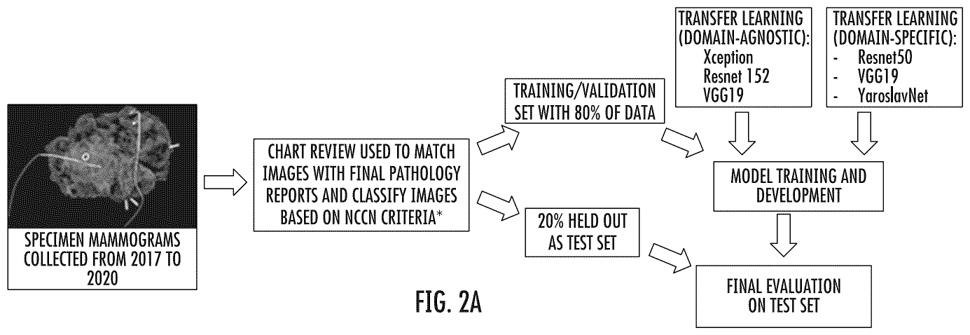

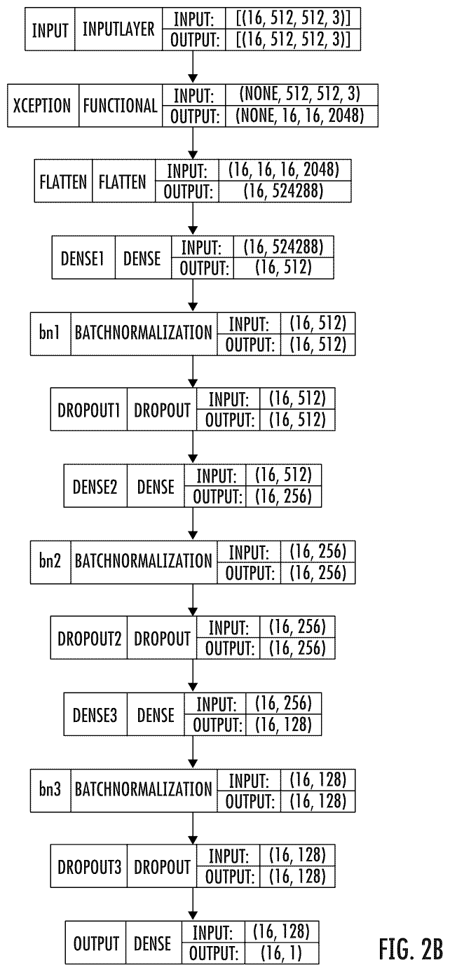

FIG. 2A shows the overall process for model development and evaluation. In particular, FIG. 2A shows specimen mammograms being matched with final pathology reports and classified based on NCCN criteria. 80% the classified mammograms were then used as a training set to train three different transfer learning models, Xception, Resnet 152, and VGG19. 20% of the classified mammograms were then used as test set to evaluate performances of each of the trained models. The models were implemented using Python (version 3.9) and the Scikitlearn and Keras libraries.

FIG. 2B shows the model architecture for the Xception-based model, which starts with the input layer, which accepts specimen mammogram images compressed and cropped to 512×512 pixels, and converts them to tensors, which are loaded onto the GPU. The Xception model is then used for feature extraction. The Xception model is a convolutional neural network trained on Internet images and that uses depth-wise separable convolutions. The “flatten” layer converts the extracted features from the Xception layers into a 1-dimensional array which is subsequently processed by additional dense layers, labeled dense1-dense3 in FIG. 2B. Although three dense layers are illustrated in FIG. 2B, more or fewer than three dense layers may be used without departing from the scope of the subject matter described herein. A dense layer is a layer of neurons where every neuron receives input from every neuron of the previous layer. The dense layers in FIG. 2B are used to classify output from the convolutional layers of the Xception model. The dense layers are trained solely on mammograms of resected breast tissue (versus the Xception model, which is initially pre-trained on Internet images and subsequently trained with the dense layers on mammograms of resected breast tissue). Batch normalization and dropout layers are added after each dense layer to improve model training and improve model generalizability, respectively. The dropout layers randomly remove the connection between neurons in the dense layers to prevent overfitting. Finally, the output layer with 1 neuron expresses the model prediction, which is a probability between 0 and 1 of whether an image depicts a positive tumor margin.

### Results

The dataset included a total of 450 images. 208 were classified as “positive” and 242 as “negative.” Within the positive classification, 63 had mixed pathology with both invasive cancer and DCIS 2 mm from the margin. After splitting into training and validation groups, 315 images were used for training and 135 were used for testing. The validation group included 72 positive images and 62 negative images.

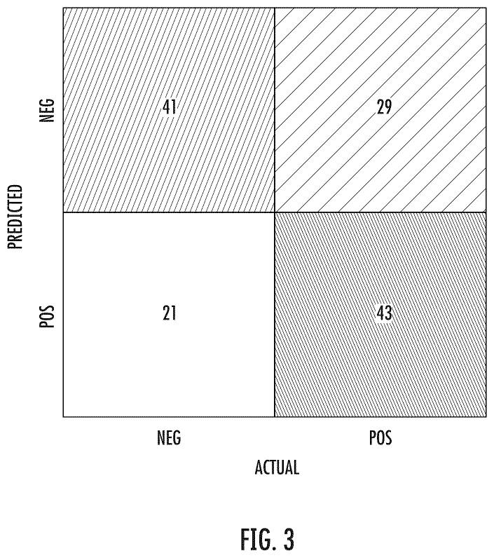

The Xception-based model showed the best performance. After 6 total epochs of training, the model achieved a balanced accuracy of 68%. For positive margins, the sensitivity was 69%, while the specificity was 66%. The positive predictive value was 60% and the negative predictive value was 66%. FIG. 3 is a confusion matrix illustrating predictive ability of the trained Xception model for positive and negative margin statuses;

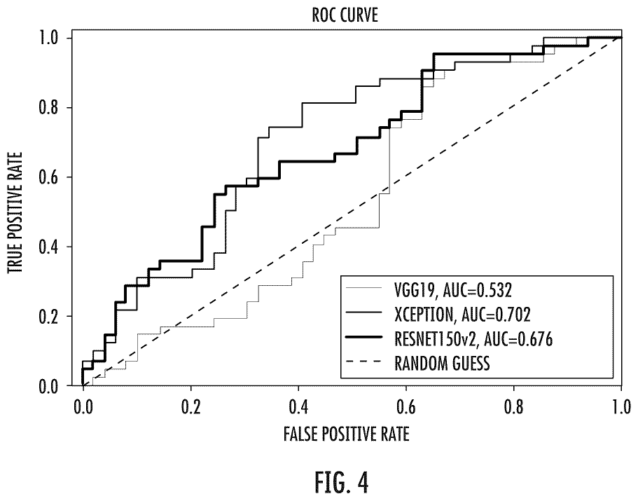

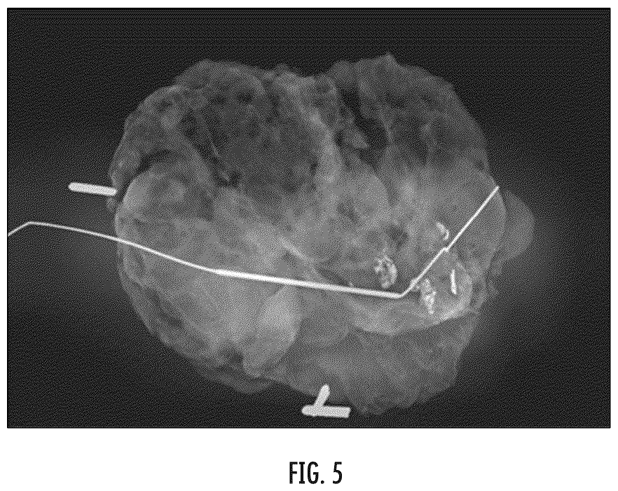

The area under the receiver operating characteristic curve was 0.72. Receiver operating characteristics curves for Xception and the other models are shown in FIG. 4. Pixel importance analysis using GradCam showed that the model was correctly identifying the area of tumor as highly important for decision-making, as opposed to extraneous features (FIG. 5).

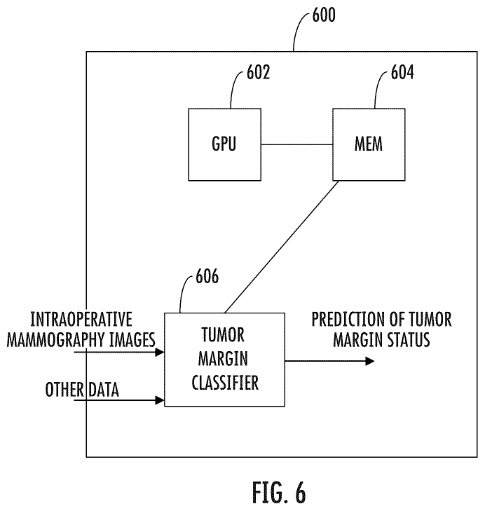

FIG. 6 is a block diagram illustrating a computing platform including a tumor margin classifier comprising a trained machine learning model for intra-operative assessment of tumor margin status. In FIG. 6, a computing platform on which the tumor margin classifier executes. Referring to FIG. 6, computing platform 600 may be any suitable computing platform that can run in an operating room. Computing platform 600 may include a graphics processing unit (GPU) 602 and memory 604. Computing platform 600 may include a tumor margin classifier 606 comprising a trained machine learning algorithm, for example, as illustrated in FIG. 2B. Tumor margin classifier 606 may be implemented using computer executable instructions stored in memory 604 and executed by GPU 602. Tumor margin classifier 606 receives intra-operative mammography images, passes the images through the layers of the trained neural network illustrated in FIG. 2B, and outputs a prediction of tumor margin status, which in one example, is a number between 0 and 1 indicating a probability or likelihood that the specimen has a positive tumor margin status. In an alternate implementation, tumor margin classifier 606 could be configured to output a probability or likelihood that the specimen has a negative tumor margin status.

Tumor margin classifier 606 may also receive input from source in addition to mammograms to enhance the predictive accuracy of the output. These other sources are illustrated in FIG. 6 by the “other data” input. Examples of other sources of data that may be input to tumor image classifier 606 to enhance the predictive accuracy include clinical data, genetic data, image data other than mammograms (e.g., MR or CT image data), specimen tomosynthesis data (which are 3D x-ray images of breast tissue specimens), etc. In order to process the different data types, the trained neural network implemented by tumor image classifier 606 may be branched, with one branch trained to classify data and generate an output indicative of tumor margin status for the given data type. The outputs from the branches would be combined by a layer that produces an output indicative of tumor margin status based on the combined outputs of the branches.

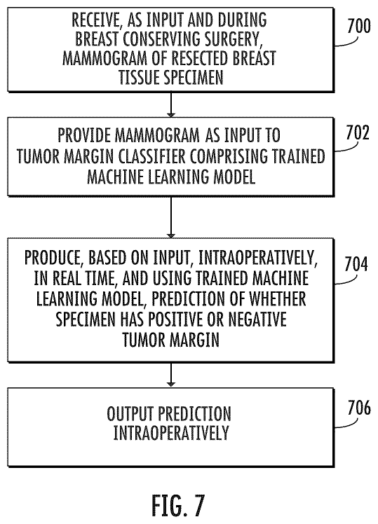

FIG. 7 is a flow chart illustrating an exemplary process for intra-operative assessment of tumor margin status. Referring to FIG. 7, in step 700, the process includes receiving, as input and during breast-conserving surgery, a mammogram of a resected breast tissue specimen. For example, resected human breast tissue specimen mammograms generated by a specimen mammography imaging system can be received in digital format by a computing system in an operating room. The computing system may be integrated within the specimen mammography imaging system or separate from the specimen mammography imaging system. If the computing system is separate from the specimen mammography imaging system, the mammograms may be transmitted to the computing system via a wired or wireless connection, or, alternatively, via a removable storage medium, such as a thumb drive.

In step 702, the process includes providing the mammogram as input to a tumor margin classifier comprising a trained machine learning model. For example, the mammogram of the resected breast tissue specimen may be input to tumor margin classifier 606, which implements the machine learning model illustrated in FIG. 2B. The machine learning model includes a trained image classifier model, such as the Xception model, that was pre-trained using at least some non-mammography images, such as images of people and objects available on the Internet. The machine learning model also includes at least one dense layer that was initially trained while the layers of the Xception model were frozen using mammograms of resected breast tissue and pathologic margin statuses determined by experts. The dense layers and the layers of the Xception model were then trained together (with the layers of the Xception model unfrozen) using mammograms of resected breast tissue and pathologic margin statuses determined by experts. Keeping the model frozen during initial training means that weights assigned to the neurons in the frozen layers do not change during the training. Thus, the weights assigned to the neurons in the dense layers result from both the initial training while the Xception model was frozen and subsequent training in combination with the Xception model with the Xception model unfrozen.

In step 704, the process includes producing, based on the input, intra-operatively, in real time, and using the trained machine learning model, a prediction of whether the specimen has a positive tumor margin status or a negative tumor margin status. For example, tumor margin classifier 606 may produce an output based on the intra-operative resected breast tissue mammogram, which in one example, is a number between 0 and 1 indicating a likelihood that the specimen has a positive margin status. Alternatively, the output could be modified to indicate a likelihood that the specimen has a negative margin status.

In step 706, the process includes outputting the prediction intra-operatively. For example, tumor margin classifier 606 may visually output on a computer display screen, the prediction of margin status in real time (e.g., on the order of seconds after the image data is input to tumor margin classifier 606) to the surgeon during surgery so that the surgeon can decide whether additional breast tissue needs to be removed or whether the surgery is complete and no further tissue removal is required. Presenting a margin status prediction in real time intra-operatively where the margin status prediction is produced by a machine learning model trained both using non-mammography images and using mammograms labeled with pathologic margin status is advantageous over relying solely on visual classification of margin statuses by humans, who may or may not be experts in breast-conserving surgery.

### Discussion

This project developed a model which assesses the margin status of specimen mammograms with accuracy that compares favorably to human interpretation, despite use of a relatively small training set. These results indicate that the use of computer vision to predict margin status from specimen mammograms has significant potential.

Despite advances in intra-operative margin assessment, the rate of positive margin after partial mastectomy remains high.¹ Specimen mammography is a commonly used method that benefits from its availability within the operating room and its ability to provide immediate feedback.

However, the diagnostic accuracy of specimen mammography is highly variable and relatively low, with reported sensitivity ranging from 20-58%.²⁻⁵ A 2017 meta-analysis of 9 studies showed a pooled sensitivity of 53% (95% CI 45-61%) with a pooled specificity of 84% (95% CI 77-89%).¹⁴

The current model's accuracy metrics compare favorably to the published literature with a sensitivity of 69% compared with 53%.¹⁴ These findings mirror other recent advances in computer vision classification of mammograms, which show improved accuracy when radiologists are assisted by Al.¹⁵⁻¹⁷ Al has also recently been successfully applied to laparoscopic video, demonstrating its potential to assist with real-time, intra-operative decision-making.¹⁸⁻²⁰

This project has several limitations. The accuracy and generalizability of the model is most significantly limited by the size and homogeneous (single-surgeon, single-institution) nature of the dataset. A larger, multi-institution dataset would create a more accurate and robust model and verify its external validity. 3D tomosynthesis may also be more accurate compared with specimen mammography and result in improved models.²¹⁻²³ In addition, the model does not identify which margin may be involved. Use of explainability techniques to localize features that suggest a positive margin is possible and represents a target for future research.^{24,25} Finally, we did not assess radiologist or surgeon performance for classification on our dataset, although this is likely to be similar to that seen in the literature.

In conclusion, we present work on a prototype model that can predict the pathologic margin status of partial mastectomy specimens based on intra-operative specimen mammograms. The model's predictions compare favorably with human interpretation. Optimized versions of this model could reduce the rate of positive margins and need for re-operation in breast-conserving surgery.

### Proposed Model for Predicting Tumor Margins from 3D Tomosynthesis Data

As indicated above, the model described herein can be used to predict tumor margins from 2D mammograms of resected breast tissue. It is believed that the model can be extended to predict tumor margins from 3D mammograms, also referred to as 3D radiographs, of resected breast tissue. The process of obtaining 3D radiographs of resected breast tissue is referred to as specimen tomosynthesis. In specimen tomosynthesis, 20-30 X-rays are taken of each specimen at 1 mm intervals. Together, these can provide a more complete image of the tumor and its margins. We plan to apply a more advanced modeling technique to predict pathologic margin status based on these images. This model will use the same computer vision neural network described above to convert each 1 mm image slice into feature-rich embeddings. These embeddings will then be processed by a transformer model, which processes sequential data. Finally, domain-specific classification layers will process the output of the transformer model for the final margin prediction.

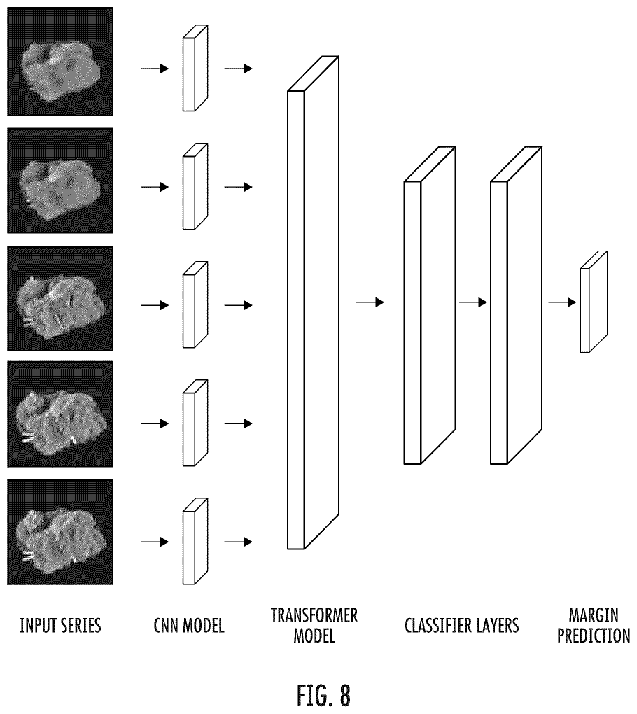

FIG. 8 is a diagram illustrating a proposed model for predicting tumor margins from 3D tomosynthesis data. In FIG. 8, the images on the left-hand side are images of 1 mm thick slices of a 3D image of resected breast tissue. The CNN model converts the image slices into the feature-rich embeddings. The CNN model may have the same structure as the Xception layer described above with regard to FIG. 2B. The transformer model is a sequential or time series model that considers relationships between the embeddings produced by the CNN model. The output of the transformer model is a vector of values indicating the relative importance of the feature embeddings output from the CNN. The remaining predictive and classification layers will be trained using pathologist-generated predictions of tumor margin status from the 3D tomosynthesis data as ground truth data. The result will be a trained model that outputs a prediction of tumor margin status from 3D tomosynthesis data.

The disclosure of each of the following references is hereby incorporated herein by reference in its entirety.

### REFERENCES

1. Morrow M, Abrahamse P, Hofer TP, et al. Trends in Reoperation After Initial Lumpectomy for Breast Cancer Addressing Overtreatment in Surgical Management Author Audio Interview Supplemental content. JAMA Oncol. 2017;3(10):1352-1357. doi:10.1001/jamaoncol.2017.0774
2. Layfield DM, May DJ, Cutress RI, et al. The effect of introducing an in-theatre intra-operative specimen radiography (IOSR) system on the management of palpable breast cancer within a single unit. Breast. 2012;21(4):459-463. doi:10.1016/j.breast.2011.10.010
3. Bathla L, Harris A, Davey M, Sharma P, Silva E. High resolution intra-operative two-dimensional specimen mammography and its impact on second operation for re-excision of positive margins at final pathology after breast conservation surgery. Am J Surg. 2011;202:387-394. doi:10.1016/j.amjsurg.2010.09.031
4. Hisada T, Sawaki M, Ishiguro J, et al. Impact of intra-operative specimen mammography on margins in breast-conserving surgery. Mol Clin Oncol. 2016;5(3):269-272. doi:10.3892/mco.2016.948
5. Funk A, Heil J, Harcos A, et al. Efficacy of intra-operative specimen radiography as margin assessment tool in breast conserving surgery. Breast Cancer Res Treat. 2020;179(2):425-433. doi:10.1007/s10549-019-05476-6
6. Yala A, Lehman C, Schuster T, Portnoi T, Barzilay R. A Deep Learning Mammography-based Model for Improved Breast Cancer Risk Prediction. Radiology. 2019;292(1):60-66. doi:10.1148/radiol.2019182716
7. Lehman CD, Yala A, Schuster T, et al. Mammographic breast density assessment using deep learning: Clinical implementation. Radiology. 2019;290(1):52-58. doi:10.1148/radiol.2018180694
8. Hashimoto DA, Rosman G, Witkowski ER, et al. Computer Vision Analysis of Intra-operative Video: Automated Recognition of Operative Steps in Laparoscopic Sleeve Gastrectomy. Ann Surg. July 2019. doi:10.1097/SLA.0000000000003460
9. Gradishar WJ, Anderson BO, Abraham J, et al. Breast cancer, version 3.2020. JNCCN J Natl Compr Cancer Netw. 2020;18(4):452-478. doi:10.6004/jnccn.2020.0016
10. Zheng A, Casari A. Feature Engineering for Machine Learning PRINCIPLES AND TECHNIQUES FOR DATA SCIENTISTS.
11. Russakovsky O, Deng J, Su H, et al. ImageNet Large Scale Visual Recognition Challenge. Int J Comput Vis. 2015;115(3):211-252. doi:10.1007/s11263-015-0816-y
12. Smith LN. A disciplined approach to neural network hyper-parameters: Part 1—learning rate, batch size, momentum, and weight decay. March 2018. http://arxiv.org/abs/1803.09820. Accessed Jul. 27, 2020.
13. Selvaraju RR, Cogswell M, Das A, Vedantam R, Parikh D, Batra D. Grad-CAM: Visual Explanations from Deep Networks via Gradient-based Localization. Int J Comput Vis. 2016;128(2):336-359. doi:10.1007/s11263-019-01228-7
14. St John ER, Al-Khudairi R, Ashrafian H, et al. Diagnostic accuracy of intra-operative techniques for margin assessment in breast cancer surgery a meta-analysis. Ann Surg. 2017;265(2):300-310. doi:10.1097/SLA.0000000000001897
15. Kim HE, Kim HH, Han BK, et al. Changes in cancer detection and false-positive recall in mammography using artificial intelligence: a retrospective, multireader study. Lancet Digit Heal. 2020;2(3): e138-e148. doi:10.1016/S2589-7500(20)30003-0
16. Rodriguez-Ruiz A, Lång K, Gubern-Merida A, et al. Stand-Alone Artificial Intelligence for Breast Cancer Detection in Mammography: Comparison With 101 Radiologists. JNCI J Natl Cancer Inst. 2019;111(9):916-922. doi:10.1093/JNCI/DJY222
17. Rodríguez-Ruiz A, Krupinski E, Mordang J-J, et al. Detection of Breast Cancer with Mammography: Effect of an Artificial Intelligence Support System. https://doi.org/101148/radio/2018181371. 2018;290(3):305-314. doi: 10.1148/RADIOL.2018181371
18. Madani A, Namazi B, Altieri MS, et al. Artificial Intelligence for Intra-operative Guidance: Using Semantic Segmentation to Identify Surgical Anatomy During Laparoscopic Cholecystectomy. Ann Surg. 2020; Publish Ah. doi:10.1097/sla.0000000000004594
19. D K, N T, H M, et al. Real-time automatic surgical phase recognition in laparoscopic sigmoidectomy using the convolutional neural network-based deep learning approach. Surg Endosc. 2020;34(11):4924-4931. doi:10.1007/S00464-019-07281-0
20. Cheng K, You J, Wu S, et al. Artificial intelligence-based automated laparoscopic cholecystectomy surgical phase recognition and analysis. Surg Endosc 2021. 2021;1:1-9. doi:10.1007/S00464-021-08619-3
21. Partain N, Calvo C, Mokdad A, et al. Differences in Re-excision Rates for Breast-Conserving Surgery Using Intra-operative 2D Versus 3D Tomosynthesis Specimen Radiograph. Ann Surg Oncol 2020 2712. 2020;27(12):4767-4776. doi:10.1245/S10434-020-08877-W
22. Un Park K, Kuerer HM, Rauch GM, et al. Digital Breast Tomosynthesis for Intra-operative Margin Assessment during Breast-Conserving Surgery. 2019. doi:10.1245/s10434-019-07226-w
23. Polat YD, Taşkin F, Çildağ MB, Tanyeri A, Soyder A, Ergin F. The role of tomosynthesis in intra-operative specimen evaluation. Breast J. 2018;24(6):992-996. doi:10.1111/TBJ.13070
24. Ghorbani A, Wexler Google Brain J, Zou J, Kim Google Brain B. Towards Automatic Concept-Based Explanations. https://github.com/amiratag/ACE.
25. Sercan″ S, Arik S, Pfister T. Protoattend: Attention-Based Prototypical Learning.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for intra-operative assessment of a tissue specimen mammogram to predict tumor margin status, the method comprising:
receiving, during breast-conserving surgery, a mammogram of a resected breast tissue specimen;
providing the mammogram as input to a tumor margin classifier comprising a trained machine learning model;
producing, based on the input, intra-operatively, in real time, and using the trained machine learning model, a prediction of whether the specimen has a positive tumor margin status or a negative tumor margin status; and
outputting the prediction intra-operatively.

2. The method of claim 1 comprising using the prediction to determine whether to resect additional breast tissue.

3. The method of claim 1 wherein the trained machine learning model comprises an image classifier model pre-trained based on non-mammography images.

4. The method of claim 3 wherein the trained machine learning model comprises at least one dense layer connected to the image classifier model.

5. The method of claim 4 wherein weights assigned to neurons in the at least one dense layer result from training of the at least one dense layer using mammograms and expert margin status classifications as inputs with the image classifier model frozen.

6. The method of claim 5 wherein the weights assigned to neurons in the at least one dense layer additionally result from training of the at least one dense layer in combination with the image classifier model and with the image classifier model unfrozen.

7. The method of claim 4 wherein the trained machine learning model comprises at least one dropout layer following the at least one dense layer.

8. The method of claim 1 wherein the prediction of whether the specimen has a positive tumor margin status or a negative tumor margin status comprises a probability that the specimen has a positive tumor margin status.

9. The method of claim 1 wherein the prediction of whether the specimen has a positive tumor margin status or a negative tumor margin status comprises a probability that the specimen has a negative tumor margin status.

10. The method of claim 1 wherein the trained machine learning model is configured to generate the prediction of whether the specimen has a positive tumor margin status or a negative tumor margin status based on at least one input in addition to the mammogram.

11. The method of claim 1 wherein the mammogram comprises a two dimensional image of the resected breast tissue.

12. The method of claim 1 wherein the mammogram comprises a three dimensional image of the resected breast tissue.

13. A system for intra-operative assessment of a tissue specimen mammogram to predict tumor margin status, the system comprising:
a computing platform including at least one processor and a memory; and
a tumor margin classifier comprising a trained machine learning model implemented by the at least one processor for receiving as input during breast-conserving surgery, a mammogram of a resected breast tissue specimen, producing, based on the input, intra-operatively, in real time, and using the trained machine learning model, a prediction of whether the specimen has a positive tumor margin status or a negative tumor margin status, and outputting the prediction intra-operatively.

14. The system of claim 13 wherein the trained machine learning model comprises an image classifier model pre-trained based on non-mammography images.

15. The system of claim 14 wherein the trained machine learning model comprises at least one dense layer connected to the image classifier model.

16. The system of claim 15 wherein weights assigned to neurons in the at least one dense layer resulted from initial training of the at least one dense layer using mammograms and expert margin status classifications as inputs with the image classifier model frozen.

17. The system of claim 16 wherein the weights assigned to the neurons in the at least one dense layer additionally result from training of the at least one dense layer in combination with the image classifier model and with the image classifier model unfrozen.

18. The system of claim 15 wherein the trained machine learning model comprises at least one dropout layer following the at least one dense layer.

19. The system of claim 13 wherein the prediction of whether the specimen has a positive tumor margin status or a negative tumor margin status comprises a probability that the specimen has a positive tumor margin status.

20. The system of claim 13 wherein the prediction of whether the specimen has a positive tumor margin status or a negative tumor margin status comprises a probability that the specimen has a negative tumor margin status.

21. The system of claim 11 wherein the trained machine learning model is configured to generate the prediction of whether the specimen has a positive tumor margin status or a negative tumor margin status based on at least one input in addition to the mammogram.

22. The system of claim 13 wherein the mammogram comprises a two dimensional image of the resected breast tissue.

23. The system of claim 13 wherein the mammogram comprises a three dimensional image of the resected breast tissue.

24. A non-transitory computer readable medium having stored thereon executable instructions that when executed by a processor of a computer control the computer to perform steps comprising:
receiving, during breast-conserving surgery, a mammogram of a resected breast tissue specimen;
providing the mammogram as input to a tumor margin classifier comprising a trained machine learning model;
producing, based on the input, intra-operatively, in real time, and using the trained machine learning model, a prediction of whether the specimen has a positive tumor margin status or a negative tumor margin status; and
outputting the prediction intra-operatively.
